(19) United States
(12) Patent Application Publication
(10) Pub. No.: US 2012/0276030 A1
(43) Pub. Date: Nov. 1, 2012

(54) COSMETIC COMPOSITION

(75) Inventors: WILLIAM J. MARTHALER, HUDSON, OH (US); CHRISTINE D. HOLMGREN, AKRON, OH (US)

(73) Assignee: GARDEN ART INNOVATIONS, LLC, AKRON, OH (US)

(21) Appl. No.: 13/220,096

(22) Filed: Aug. 29, 2011

Related U.S. Application Data
(60) Provisional application No. 61/481,115, filed on Apr. 29, 2011.

(51) Int. Cl. A61K 8/92; A61Q 1/02; A61K 8/97

(52) U.S. Cl. 424/63

(57) ABSTRACT

An anhydrous colored cosmetic composition containing a blend or mixture of a solid butter and a colorant. The anhydrous colored cosmetic composition may also include at least one oil component in addition to the solid butter and colorant. The cosmetic composition does not contain any added exogenous water. Because the anhydrous colored cosmetic composition does not contain exogenous water, no chemical emulsifiers or preservative are required to maintain the stability of the cosmetic composition.

This application claims priority to a provisional patent application filed Apr. 29, 2011, entitled ANHYDROUS COLORED COSMETIC COMPOSITION, having Ser. No. 61/481,115, which is hereby incorporated by reference. The present disclosure relates to an anhydrous colored cosmetic composition comprising at least one butter and a colorant. The anhydrous colored cosmetic composition may be used as a cosmetic or make-up product for the skin.

### TECHNICAL FIELD

### Background

Cosmetic compositions are used to beautify the skin. There are many synthetic chemicals included in cosmetic compositions to impart various properties to the cosmetic composition and to maintain the stability of the cosmetic compositions. Many of these chemicals are known to penetrate the skin and enter into the body. The penetration of such chemicals into the body remains a concern that can lead to disease. What is needed in the art is a cosmetic composition that avoids the inclusion of potentially harmful chemicals, which is based on a simple and primarily natural system, and which imparts the desired beautifying effect to the skin.

### DEFINITIONS

Anhydrous—being without water, especially water of hydration.

Butter—a triglyceride having a titer point below 40° C. (104° F.) but above 20° C. (68° F.).

Cosmetic—any substance that is rubbed, poured, sprinkled, sprayed on, introduced into, or otherwise applied to the human body or any part thereof for the purpose of beautifying, cleansing, altering, enhancing, or promoting the attractiveness of the skin, or protecting the skin.

Dimethylpolysiloxane—also referred to in the International Cosmetic and Ingredient Dictionary and Handbook as “dimethicone.” It comprises a mixture of fully methylated siloxane polymers end capped with various chemical groups. The dimethylpolysiloxane may be end capped with the same or different organic groups.

Essential oil—a concentrated hydrophobic liquid containing volatile aroma compounds from plants.

Exogenous—originating from outside; derived externally.

Fat—a triglyceride having a titer point above 40.5° C. (104.9° F.).

Log reduction—a mathematical term used to show the relative number of live microbes eliminated from a surface. 1 log reduction means the number of germs is 10 times smaller, 2 log reduction means the number of germs is 100 times smaller, 3 log reduction means the number of germs is 1,000 times smaller, 4 log reduction means the number of germs is 10,000 times smaller, 5 log reduction means the number of germs is 100,000 times smaller, 6 log reduction means the number of germs is 1,000,000 times smaller, and 7 log reduction means the number of germs is 10,000,000 times smaller.

Make-up—A subset of cosmetics, which refers primarily to colored products intended to alter the appearance of the user's skin.

Oil—a triglyceride having a titer point below 40.5° C. (104.9° F.), is liquid at room temperature, and is insoluble in water.

Phthalates—esters of phthalic acid, an aromatic dicarboxylic acid, with formula C₆H₄(CO₂H)₂.

Pigment—a material that changes the color of reflected or transmitted light as the result of wavelength-selective absorption.

Polysiloxane—(R₂SiO)ₙ A polymer in which the chain contains alternate silicon and oxygen atoms; in the formula, R can be H or an alkyl or aryl group

Synthetic dye—Any of the organic dyes originally derived from coal-tar derivatives, but currently synthesized from benzene and its derivatives.

Synthetic fragrance—non-naturally occurring aromachemicals created to emulate natural fragrances.

Vegetable oil—lipid materials derived from plants.

Wax—a class of chemical compounds that are malleable near ambient temperatures. Characteristically, they melt above 45° C. (113° F.) to give a low viscosity liquid. Waxes are insoluble in water but soluble in organic, nonpolar solvents. All waxes are organic compounds, both synthetic and naturally occurring.

### SUMMARY

Provided is an anhydrous colored cosmetic composition comprising greater than 50% by weight of at least one solid butter and a colorant, wherein the composition does not contain any exogenous water added.

Additionally provided is a process for preparing an anhydrous colored cosmetic composition comprising blending together greater than 50% by weight of at least one solid butter and a colorant, wherein no exogenous water is added during the blending.

### DETAILED DESCRIPTION

An anhydrous cosmetic composition is made by blending together at least one butter and at least one cosmetically or dermatologically acceptable colorant. In addition, at least one oil can also be added. In an illustrative embodiment, the butters, and any component that is liquid at room temperature, can be blended and folded into a homogenous mixture. Any material that is solid at room temperature is melted and poured into a mixture, or is melted into other oils and/or butters as carriers.

An anhydrous colored cosmetic composition comprising a blend or mixture of at least one solid butter component and at least one cosmetically or dermatologically acceptable colorant is disclosed. The solid butter component of the anhydrous colored cosmetic composition may comprise a butter that is derived from a vegetable, fruit, nut, or seed source.

According to certain illustrative embodiments, the anhydrous colored cosmetic composition may comprise a blend of more than one different butter and at least one cosmetically or dermatologically acceptable colorant.

According to certain illustrative embodiments, the anhydrous colored cosmetic composition comprises a blend or mixture of greater than about 50% by weight of at least one solid butter component (which includes, but is not limited to greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, and greater than 99) and at least one cosmetically or dermatologically acceptable colorant.

According to certain illustrative embodiments, the anhydrous colored cosmetic composition comprises a blend or mixture of greater than about 75% by weight of at least one solid butter component (which includes, but is not limited to greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, and greater than 99) and at least one cosmetically or dermatologically acceptable colorant.

According to certain illustrative embodiments, the anhydrous colored cosmetic composition comprises a blend or mixture of greater than about 90% by weight of at least one solid butter component (which includes, but is not limited to greater than 90, greater than 91, greater than 92, greater than 93, greater than 94, greater than 95, greater than 96, greater than 97, greater than 98, and greater than 99) and at least one cosmetically or dermatologically acceptable colorant.

According to certain illustrative embodiments, the anhydrous colored cosmetic composition comprises a blend or mixture of greater than about 95% by weight of at least one solid butter component (which includes, but is not limited to greater than 95, greater than 96, greater than 97, greater than 98, and greater than 99) and at least one cosmetically or dermatologically acceptable colorant.

According to certain illustrative embodiments, the anhydrous colored cosmetic composition comprises a blend or mixture of greater than about 99% by weight of at least one solid butter component and at least one cosmetically or dermatologically acceptable colorant.

According to certain illustrative embodiments, the anhydrous colored cosmetic composition may comprise a butter, an oil, and at least one cosmetically or dermatologically acceptable colorant.

According to certain illustrative embodiments, the anhydrous colored cosmetic composition may comprise a blend of more than one type of butter, an oil, and at least one cosmetically or dermatologically acceptable colorant. While an oil component may be included in the anhydrous colored cosmetic composition, in this embodiment, the butter component is in an amount that is greater than the amount of the oil component of the cosmetic composition.

According to certain illustrative embodiments, the anhydrous colored cosmetic composition may comprise a blend of more than one type of butter, more than one type of oil, and at least one cosmetically or dermatologically acceptable colorant.

According to certain illustrative embodiments, the anhydrous colored cosmetic composition comprises a blend or mixture of greater than about 50% by weight of at least one solid butter (which includes, but is not limited to greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, and greater than 99), at least one oil, and at least one cosmetically or dermatologically acceptable colorant.

According to certain illustrative embodiments, the anhydrous colored cosmetic composition comprises a blend or mixture of greater than about 75% by weight of at least one solid butter (which includes, but is not limited to greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, and greater than 99), at least one oil, and at least one cosmetically or dermatologically acceptable colorant.

According to certain illustrative embodiments, the anhydrous colored cosmetic composition comprises a blend or mixture of greater than about 90% by weight of at least one solid butter (which includes, but is not limited to greater than 90, greater than 91, greater than 92, greater than 93, greater than 94, greater than 95, greater than 96, greater than 97, greater than 98, and greater than 99), at least one oil, and at least one cosmetically or dermatologically acceptable colorant.

According to certain illustrative embodiments, the anhydrous colored cosmetic composition comprises a blend or mixture of greater than about 95% by weight of at least one solid butter (which includes, but is not limited to greater than 95, greater than 96, greater than 97, greater than 98, and greater than 99), at least one oil, and at least one cosmetically or dermatologically acceptable colorant.

According to certain illustrative embodiments, the anhydrous colored cosmetic composition comprises a blend or mixture of greater than about 99% by weight of at least one solid butter, at least one oil, and at least one cosmetically or dermatologically acceptable colorant.

According to certain illustrative embodiments, the anhydrous colored composition may comprise from about 45 to about 99% by weight butter (which includes, but is not limited to 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99) and from about 1 to about 65% by weight colorant (which includes, but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, and 65). According to other embodiments, the anhydrous colored composition may comprise from about 70 to about 99% by weight butter (which includes, but is not limited to 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99) and from about 1 to about 30% by weight colorant (which includes, but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30). According to additional embodiments, the anhydrous colored composition may comprise from about 80 to about 99% by weight butter (which includes, but is not limited to 80, 81, 82, 83, 84, 85, 86, 87, 88, 89, 90, 91, 92, 93, 94, 95, 96, 97, 98, and 99) and from about 1 to about 20% by weight colorant (which includes, but is not limited to 1, 2, 3, 4, 5, 6, 7, 8, 9, 10, 11, 12, 13, 14, 15, 16, 17, 18, 19, and 20).

According to certain illustrative embodiments, the anhydrous colored composition may comprise from about 45 to about 98% by weight butter (which includes, but is not limited to 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, and 98), from about 1 to about 49% by weight oil (which includes, but is not limited to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49), and from about 1 to about 54% by weight colorant (which includes, but is not limited to 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, and 54). According to other embodiments, the anhydrous colored composition may comprise from about 50 to about 80% by weight butter (which includes, but is not limited to 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80), from about 1 to about 35% by weight oil (which includes, but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and 35), and from about 1 to about 40% by weight colorant (which includes, but is not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40). According to additional embodiments, the anhydrous colored composition may comprise from about 60 to about 78% by weight butter (which includes, but is not limited to 60, 61, 62, 63, 64, 65, 66, 67, 68, 69, 70, 71, 72, 73, 74, 75, 76, 77, and 78), from about 1 to about 25% by weight oil (which includes, but is not limited to 1, 2, 3, 4, 5, 6, 7, 8, 9, 10, 11, 12, 13, 14, 15, 16, 17, 18, 19, 20, 21, 22, 23, 24, and 25), and from about 1 to about 20% by weight colorant (which includes, but is not limited to 1, 2, 3, 4, 5, 6, 7, 8, 9, 10, 11, 12, 13, 14, 15, 16, 17, 18, 19, and 20).

According to certain embodiments, the anhydrous colored composition comprises about 49.5% by weight Cupuacu Butter, about 49.5% by weight Shea Butter, and about 1% by weight red iron oxide as a colorant. Shea butter is about 40% to about 60% by weight oleic acid (which includes, but is not limited to 40, 41, 42, 43, 44, 45, 46, 47, 48, 49, 50, 51, 52, 53, 54, 55, 56, 57, 58, 59, and 60), about 20% to about 50% stearic acid (which includes, but is not limited to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50), about 3% to about 11% linoleic acid (which includes, but is not limited to 3, 4, 5, 6, 7, 8, 9, 10, and 11), about 2% to about 9% palmitic acid (which includes, but is not limited to 2, 3, 4, 5, 6, 7, 8, and 9), less than about 1% linolenic acid, and less than about 1% arachidic acid.

According to certain embodiments, the anhydrous colored composition comprises about 50% by weight Shea Butter, about 49% by weight Safflower oil, and about 1% by weight yellow iron oxide as a colorant.

According to certain embodiments, the anhydrous colored composition comprises about 90% by weight Shea Butter and about 10% by weight titanium dioxide as a colorant.

According to certain embodiments, the anhydrous colored composition comprises about 95% by weight Shea Butter and about 5% by weight titanium dioxide as a colorant.

According to certain embodiments, the anhydrous colored composition comprises about 99% by weight Shea Butter and about 1% by weight titanium dioxide as a colorant.

According to certain embodiments, the anhydrous colored composition comprises about 71% by weight Shea Butter, about 7% by weight Safflower oil, and about 22% by weight colorant grind.

According to certain embodiments, the anhydrous colored composition comprises about 30% by weight Capuaco Butter, about 30% by weight Shea Butter, about 20% by weight Safflower oil, and about 20% by weight colorant grind.

According to certain embodiments, the anhydrous colored composition comprises about 30% by weight Capuaco Butter, about 30% by weight Shea Butter, about 10% by weight Safflower oil, about 10 Sunflower oil, and about 20% by weight colorant grind.

According to certain embodiments, the anhydrous colored composition comprises about 30% by weight Capuaco Butter, about 20% by weight Shea Butter, about 15% by weight Safflower oil, about 15 Sunflower oil, and about 20% by weight colorant grind.

According to certain embodiments, the anhydrous colored composition comprises about 90% by weight Shea Butter, about 9% by weight Sunflower oil, and about 1% by weight red iron oxide as a colorant.

According to certain embodiments, the anhydrous colored composition comprises about 95% by weight Shea Butter, about 4% by weight Sunflower oil, and about 1% by weight red iron oxide as a colorant.

According to certain embodiments, the anhydrous colored composition comprises about 99% by weight Shea Butter, about 0.5% by weight Sunflower oil, and about 0.5% by weight red iron oxide as a colorant.

According to certain embodiments, the anhydrous colored foundation comprises 69.10% Shea butter, 3.5% sunflower oil, 24.4% color phase, 0.5% grape seed oil, 0.5% jojoba oil, and 2.0% berry wax. Sunflower oil is about 4% to about 9% palmitic acid (which includes, but is not limited to 4, 5, 6, 7, 8, and 9), about 1% to about 7% stearic acid (which includes, but is not limited to 1, 2, 3, 4, 5, 6, and 7), about 10% to about 14% oleic acid (which includes, but is not limited to 10, 11, 12, 13, and 14), and about 48% to about 74% linoleic acid (which includes, but is not limited to 48, 49, 50, 51, 52, 53, 54, 55, 56, 57, 58, 59, 60, 61, 62, 63, 64, 65, 66, 67, 68, 69, 70, 71, 72, 73, and 74). Grape seed oil is about 7% palmitic acid, about 4% stearic acid, about 16% oleic acid, less than about 1% alpha-linoleic acid, less than about 1% palmitoleic acid, about 0.8% to about 1.5% tocopherols and steroids, about 72% linoleic acid, and trace amounts of vitamin E. Jojoba oil is about 66% to about 71% eicosenoic acid (which includes, but is not limited to 66, 67, 68, 69, 70, and 71), about 14% to about 20% docosenoic acid (which includes, but is not limited to 14, 15, 16, 17, 18, 19, and 20), and about 10% to about 13% oleic acid (which includes, but is not limited to 10, 11, 12, and 13), and wax esters with 36 to 46 carbon atoms. In one embodiment, the color phase is about 45% to about 50% by weight (which includes, but is not limited to 45, 46, 47, 48, 49, and 50) silicone oil and about 50% to about 55% (which includes, but is not limited to 50, 51, 52, 53, 54, and 55) by weight colorant. In one embodiment, the colorant is 2.89% black iron oxide, 5.55% red iron oxide, 17.84% yellow iron oxide, and 73.72% titanium dioxide.

According to certain embodiments, an anhydrous night cream comprises 89% Shea butter, 7.4% sunflower oil, and 3.6% colorant.

With respect to the several embodiments, a stability test was performed, which indicated two to three years of stability. The specific gravity is 0.99±0.1, the viscosity at 80° F., run on a Brookfield spindle 7 speed 10 is 20,000 to 108,000 centipoise. A temperature increase decreases the viscosity. Mixing or moving the composition decreases the viscosity to 40,000 to 60,000 centipoise. A preservative challenge test was also run. For bacteria, the pass criteria is at least a 3 log reduction (with respect to an initial count at time 0) after seven days, and no increase thereafter. For yeast and mold, the pass criteria is at least a 2 log reduction (with respect to an initial count at time 0) after fourteen days, and no increase thereafter. The composition had a 4 log reduction for bacteria and a 2 log reduction for yeast and mold.

According to certain embodiments, the anhydrous colored composition comprises a first compound comprising oleic acid, stearic acid, linoleic acid, palmitic acid, and linolenic acid, a second compound comprising linoleic acid, oleic acid, palmitic acid, stearic acid, and linolenic acid, and a colorant.

Without limitation, suitable butters that may be used in the preparation of the anhydrous colored cosmetic composition include, Aloe, Apricot, Avocado, Babassu, Bacuri, Cocoa, Coffee, Cupuacu, Drago, Hemp Seed, Illipe, Kokum, Macadamia Nut, Mango, Maaracuja, Mjochaccino, Murumuru, Olive, Pataua, Pistachio Nut, Shea, Shealaoe, Sweet Almond, Ucuuba, Wheatgerm, Tucuma, Lamond, Brazil Nut, Jojoba, Pumpkin, Soy, Chamomile, Horestail, Lavender, Lemon Grass, Matha Grean Tea, Nanoni, Lemon, Orange, Tangerine, Pam, Acai, Blueberry, Cranberry, and Pomegranate. It should be noted that the anhydrous colored cosmetic composition may comprise a single butter or may comprises a combination of more than one different type of butter.

The anhydrous colored composition may include a wax material. Without limitation, suitable waxes that may be used in the preparation of the anhydrous colored cosmetic composition include, Carnauba Wax, Jojoba (Ester) Oil, Candelilla Wax, Champaca, Jasmine, Grandiflorum, Jasmine, Sambac, Lotus, Mimosa, Rose, Tuberose, emulsifying wax, Beeswax, and the like. It should be noted that the anhydrous colored cosmetic composition may comprise a single wax or may comprise a combination of more than one different type of wax.

Without limitation, suitable essential oils that may be used in the preparation of the anhydrous colored cosmetic composition include, Allspice, Jamaica, Amyris, Angelica Root, Anise Seed, Armoise, Basil, Bay, Bergamot, Black Pepper, Cajeput, Camphor, Cananga, Cardamom, Carrot Seed, Cassia, Cedarleaf, Cedarwood, Celery Seed, Cinnamon Leaf, Cistus. Citronella, Clary Sage, Clovebud, Coriander, Cypress, Davana, Dill Seed, Elemi, Chamomile, Eucalyptus Globulus, Fennel, Sweet, Fir, Balsam, Frankincense, Galbanum, Garlic, Geranium, Ginger, Grapefruit, White, Gurjum Balsam, Helichrysum Italicumn, Hyssop, Juniper Berry, Lavandin, Lavender, Lemon, Lemongrass, Lime, Litsea Cubeba, Lovage, Mandarin, Marjoram, Mullein, Myrrh, Neroli, Niaouli, Nutmeg, Olibanum, Opoponax, Orange, Palmarosa, Patchouli, Pennyroyal, Peppermint, Petitgrain, Pine Needle, Roman Chamomile, Rose, Rosemary, Rosewood, Sage, Sandalwood, Spearmint, Spikenard, Star Anise, Tangerine, Tea Tree, Thyme, Tulsi (Holy Basil), Valencia, Verbena, Vetiver, Ylang Ylang, Zdravetz, Vanilla, Benzoin, and Tea Tree. It should be noted that the anhydrous colored cosmetic composition may comprise a single oil or may comprise a combination of more than one different type of essential oil.

Without limitation, suitable vegetable oils that may be used in the preparation of the anhydrous colored cosmetic composition include, Acai, Black Raspberry Seed, Blackberry Seed, Blackcurrant Seed, Blueberry Seed, Cranberry Seed, Red Raspberry Seed, Seabuckthorn, Strawberry Seed, Algae, Almond, Aloe Vera Extract, Andiroba, Apple Seed, Apricot Kernel, Argan, Arnica, Avocado, Baobab, Black Cumin Seed (Blackseed), Borage, Brazil Nut, Broccoli Seed, Buriti Fruit, Calendula Extract, Camellia, Carrot Extract, Castor, Chardonnay Grapeseed, Chaulmoogra, Cherry Kernel, Chilean Hazelnut (Gevuina), Cinnamon Leaf, Cocoa (Cacao), Coconut, Coffee, Copaiba Balsam, Corn, Cucumber Seed, Curry Leaf, Evening Primrose, Flax Seed, Fractionated Coconut, Grape Seed, Hazelnut, Hemp Seed, Jojoba, Karanja, Kiwi Seed, Kukui Nut, Macadamia Nut, Macauba, Mango Manketti, Maracuja (Passion Flower), Marula, Meadowfoam, Monoi de Tahiti, Moring a, Neem, Olive, Palm Kernel, Palm, Papaya, Passionfruit Oil, Pataua, Peach Kernel, Pecan, Pequi, Perilla Seed, Pomace Olive, Poppy Seed, Pracaxi, Pumpkin Seed, Red Palm Fruit, Rice Bran, Riesling Grapeseed, Rosehip, Safflower, Sangre de Drago, Saw Palmetto, Sesame, Shea, Soybean, St. John's Wort, Sunflower, Tamanu (Foraha Oil), Tomato Seed, Turkey Red, Vegetable Squalene, Vitamin E, Walnut, Watermelon Seed, Wheatgerm, Yangu, and Coconut Oil. It should be noted that the anhydrous colored cosmetic composition may comprise a single vegetable oil or may comprise a combination of more than one different type of vegetable oil.

According to certain embodiments, the anhydrous colored cosmetic composition consists essentially of at least one solid butter and a colorant, wherein the composition does not contain any exogenous water added. The process for preparing this embodiment of the anhydrous colored cosmetic composition comprises blending together ingredients consisting essentially of at least one solid butter and a colorant, wherein no exogenous water is added during the blending.

According to certain embodiments, the anhydrous colored cosmetic composition comprises at least one solid butter and a colorant, wherein the composition does not contain any exogenous water added and excludes the fatty acid ester octyldodecyl neopentanoate. The process for preparing this embodiment of the anhydrous colored cosmetic composition comprises blending together ingredients consisting essentially of at least one solid butter and a colorant, wherein no exogenous water or octyldodecyl neopentanoate is added during the blending.

The anhydrous colored composition may also include ingredients that are commonly added to cosmetic compositions for example, and without limitation, powders, emollients, conditioners, and the like.

The colorant to be added to the cosmetically acceptable component to prepare the colored cosmetic composition may be a pigment that is dispersed within a polysiloxane. When used herein, the term “the pigment” refers to a single species of pigment or a mixture of two or more different species of pigments. Therefore, it should be noted that any given cosmetic composition may include more than one different species of pigment. According to alternative embodiments, the anhydrous cosmetic composition may be prepared by blending together at least a solid fatty acid and a silicone pigment dispersion including one or more different species of pigment.

It should be noted that more than one silicone dispersion, each having one or more pigment dispersed therein, may be added to the cosmetic component to prepare a colored cosmetic composition.

According to certain illustrative embodiments, the pigment is dispersed within a polysiloxane. Without limitation, suitable polysiloxanes include organopolysiloxanes. By way of illustration, the organopolysiloxane may comprise the following chemical structure:

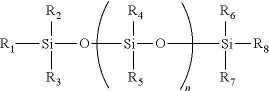

wherein R₁-R₇ are each independently selected from H, —CH₃, —CH₂CH₃, —OH, vinyl, and any other chemical group or combinations of different chemical groups which would provide a cosmetically or dermatologically acceptable polysiloxane, such as those polysiloxanes listed in the International Cosmetic and Ingredient Dictionary and Handbook; and wherein n is an integer from about 3 to about 1,000,000.

According to certain embodiments, the pigment may be dispersed in a dimethylpolysiloxane of the following structure:

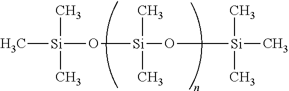

Without limitation, and only by way of exemplification, the dimethylpolysiloxane may be end capped with trialkylsiloxy groups. For example, the dimethylsiloxane groups may be end capped with a trimethylsiloxy group, a triethylsiloxy group or combinations thereof.

Without limitation, suitable examples of inorganic pigments include red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues, Lake pigments and the like.

According to certain illustrative embodiments, the pigments that may be incorporated into the dispersion include the iron oxide pigments, such as red iron oxide, yellow iron oxide, black iron oxide, brown iron oxide, titanium dioxide and mixtures thereof.

Without limitation, suitable examples of organic pigments include those which are FD&C Blue #1, FD&C Yellow #5, FD&C Yellow #6, FD&C Yellow #7, FD&C Yellow #8, FD&C Yellow #10, FD&C Red #2, FD&C Red #4, FD&C Red #6, FD&C Red #7, FD&C Red #9, FD&C Red #17, FD&C Red #19, FD&C Red #21, FD&C Red #22, FD&C Red #27, FD&C Red #28, FD&C Red #30, FD&C Red #31, FD&C Red #33, FD&C Red #34, FD&C Red #36, FD&C Red #40, D&C Violet #2, Ext D&C Violet #2, FD&C Green #3, D&C Green #5, D&C Green #6, D&C Green #8, D&C Orange #4, D&C Orange #5, D&C Brown #1, D&C Black #2, carmine and mixtures thereof.

According to other embodiments, the colorants included in the cosmetic composition may be provided by grinding them into castor oil, sorbitol, mineral oil, propylene glycol, glycerin, Jojoba oil, and the like. According to other embodiments, the colorant may be included in the cosmetic composition as a powder colorant that has been blended or mixed into the composition.

The disclosed anhydrous colored cosmetic composition is a formulation that does not contain any emulsifiers, gums, or other synthetic thickeners, yet due to its naturally creamy solid butter properties, the composition remains thick enough to hold the colorants in a homogenous product. The composition also does not include cyclic methylsiloxanes, gluten, paraben, talc, formaldehyde, mineral oil, petroleum, phthalates, sodium lauryl sulfate, synthetic dye, or synthetic fragrance. The composition is also EU Cosmetics Directive compliant.

The anhydrous composition may be used to formulate a wide variety of cosmetics. Without limitation, the composition may be used to prepare base, concealers, foundations in liquid, cream, mousse or powder forms, bronzers, face powders, face illuminators, lipstick, lip gloss, lip liner, lip plumper, lip luster, lip conditioners, lip boosters, rouge, blush (also referred to as blushers), mascara, lash extenders, lash conditioners, eyebrow liners, eye shadow, eye shimmer, eye glitter, eye pencils, eyebrow creams, eyebrow waxes, eyebrow gels, and eyebrow powders, nail polish to color fingernails and toenails.

The anhydrous composition may be used to prepare a wide variety of pigmented skin care products. Without limitation, the pigment dispersions may be used to formulate face and body creams, lotions, moisturizers, ointments, suntan oils, suntan lotions, suntan sprays, sun blocks, and sun screens.

The anhydrous composition may also be used to formulation bath soaps, bath and shower gels, bath oils, bath salts, bubble baths, bath butters, hair shampoos, hair conditioners, hair gels, hair mousses, and hair colorants.

It is to be understood that in the embodiments disclosed, the butter can be between about 45% by weight and about 99% by weight, the colorant can be between about 0.05% by weight and about 65% by weight, and the oil can be between 0.5% by weight and 49% by weight.

While the anhydrous colored cosmetic composition and process for preparing the same have been described in connection with various illustrative embodiments, it is to be understood that other similar embodiments may be used or modifications and additions may be made to the described embodiments for performing the same function disclosed herein without deviating therefrom. The embodiments described above are not necessarily in the alternative, as various embodiments may be combined to provide the desired characteristics. Therefore, the anhydrous cosmetic composition and process for preparing the same should not be limited to any single embodiment, but rather construed in breadth and scope in accordance with the recitation of the appended claims.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented hereinabove. Rather, what is intended to be covered is within the spirit and scope of the appended claims.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The invention has been described with reference to several embodiments. Obviously, modifications and alterations will occur to others upon a reading and understanding of the specification. It is intended by applicant to include all such modifications and alterations insofar as they come within the scope of the appended claims or the equivalents thereof.

Having thus described the invention, it is now claimed:

We claim:

1. A cosmetic composition comprising:
at least one butter; and,
a colorant, wherein the composition has substantially no exogenous water.

2. The composition of claim 1, wherein the at least one solid butter is at least 50% by weight.

3. The composition of claim 1, wherein the at least one butter is at least 90% by weight.

4. The composition of claim 1, wherein the at least one butter is between about 45% by weight and about 99% by weight, and the colorant is between about 0.05% by weight and 65% by weight.

5. The composition of claim 4, wherein the at least one butter is between about 70% by weight and about 99% by weight, and the colorant is between about 1% by weight and 30% by weight.

6. The composition of claim 5, wherein the at least one butter is between about 80% by weight and about 99% by weight, and the colorant is between about 1% by weight and 20% by weight.

7. The composition of claim 1, wherein the colorant comprises a pigment dispersion.

8. The composition of claim 7, wherein the pigment dispersion comprises a polysiloxane, wherein the polysiloxane comprises the following chemical structure: 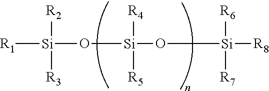
wherein R₁-R₇ are each independently selected from H, —CH₃, —CH₂CH₃, —OH, vinyl; and,
wherein n is an integer from about 3 to about 1,000,000.

9. The composition of claim 8, wherein the polysiloxane is dimethylpolysiloxane.

10. The composition of claim 9, wherein the pigment comprises an inorganic pigment, an organic pigment, or mixtures thereof.

11. The composition of claim 10, wherein the pigment comprises an inorganic pigment selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

12. The composition of claim 10, wherein the pigment comprises an organic pigment selected from the group consisting of FD&C Blue #1, FD&C Yellow #5, FD&C Yellow #6, FD&C Yellow #7, FD&C Yellow #8, FD&C Yellow #10, FD&C Red #2, FD&C Red #4, FD&C Red #6, FD&C Red #7, FD&C Red #9, FD&C Red #17, FD&C Red #19, FD&C Red #21, FD&C Red #22, FD&C Red #27, FD&C Red #28, FD&C Red #30, FD&C Red #31, FD&C Red #33, FD&C Red #34, FD&C Red #36, FD&C Red #40, D&C Violet #2, Ext D&C Violet #2, FD&C Green #3, D&C Green #5, D&C Green #6, D&C Green #8, D&C Orange #4, D&C Orange #5, D&C Brown #1, D&C Black #2, carmine, and mixtures thereof.

13. The composition of claim 1, wherein the composition further comprises an oil.

14. The composition of claim 13, wherein the at least one butter is between about 45% by weight and about 98% by weight, the oil is between about 1% by weight and about 49% by weight, and the colorant is between about 0.05% by weight and 54% by weight.

15. The composition of claim 14, wherein the at least one butter is between about 50% by weight and about 80% by weight, the oil is between about 1% by weight and about 35% by weight, and the colorant is between about 1% by weight and 40% by weight.

16. The composition of claim 15, wherein the at least one butter is between about 60% by weight and about 78% by weight, the oil is between about 1% by weight and about 25% by weight, and the colorant is between about 1% by weight and 20% by weight.

17. The composition of claim 14, wherein the butter is chosen from the group comprising aloe, apricot, avocado, babassu, bacuri, cocoa, coffee, cupuacu, drago, hemp seed, illipe, kokum, macadamia nut, mango, maaracuja, mjochaccino, murumuru, olive, pataua, pistachio nut, shea, shealaoe, sweet almond, ucuuba, wheatgerm, tucuma, lamond, brazil nut, jojoba, pumpkin, soy, chamomile, horestail, lavender, lemon grass, matha green tea, nanoni, lemon, orange, tangerine, pam, acai, blueberry, cranberry, pomegranate, and mixtures thereof,
wherein the oil is chosen from the group comprising allspice, jamaica, amyris, angelica root, anise seed, armoise, basil, bay, bergamot, black pepper, cajeput, camphor, cananga, cardamom, carrot seed, cassia, cedarleaf, cedarwood, celery seed, cinnamon leaf, cistus, citronella, clary sage, clovebud, coriander, cypress, davana, dill seed, elemi, chamomile, eucalyptus globulus, fennel, sweet, fir, balsam, frankincense, galbanum, garlic, geranium, ginger, grapefruit, white, gurjum balsam, helichrysum italicum, hyssop, juniper berry, lavandin, lavender, lemon, lemongrass, lime, litsea cubeba, lovage, mandarin, marjoram, mullein, myrrh, neroli, niaouli, nutmeg, olibanum, opoponax, orange, palmarosa, patchouli, pennyroyal, peppermint, petitgrain, pine needle, roman chamomile, rose, rosemary, rosewood, sage, sandalwood, spearmint, spikenard, star anise, tangerine, tea tree, thyme, tulsi, valencia, verbena, vetiver, ylang ylang, zdravetz, vanilla, benzoin, tea tree, acai, black raspberry seed, blackberry seed, blackcurrant seed, blueberry seed, cranberry seed, red raspberry seed, seabuckthorn, strawberry seed, algae, almond, aloe vera extract, andiroba, apple seed, apricot kernel, argan, arnica, avocado, baobab, black cumin seed, borage, brazil nut, broccoli seed, buriti fruit, calendula extract, camellia, carrot extract, castor, chardonnay grapeseed, chaulmoogra, cherry kernel, chilean hazelnut, cinnamon leaf, cocoa, coconut, coffee, copaiba balsam, corn, cucumber seed, curry leaf, evening primrose, flax seed, fractionated coconut, grape seed, hazelnut, hemp seed, jojoba, karanja, kiwi seed, kukui nut, macadamia nut, macauba, mango manketti, maracuja, marula, meadowfoam, monoi de tahiti, moringa, neem, olive, palm kernel, palm, papaya, passionfruit oil, pataua, peach kernel, pecan, pequi, perilla seed, pomace olive, poppy seed, pracaxi, pumpkin seed, red palm fruit, rice bran, riesling grapeseed, rosehip, safflower, sangre de drago, saw palmetto, sesame, shea, soybean, St. john's wort, sunflower, tamanu, tomato seed, turkey red, vegetable squalene, vitamin E, walnut, watermelon seed, wheatgerm, yangu, coconut oil, and mixtures thereof.

18. A process for preparing a cosmetic composition comprising the steps of:
blending together greater than 50% by weight of at least one butter and a colorant, wherein the composition contains substantially no exogenous water.

19. The process of claim 18, wherein the at least one butter is at least 90% by weight.

20. The process of claim 19, wherein the at least one butter is between about 45% by weight and about 99% by weight, and the colorant is between about 0.05% by weight and 65% by weight.

21. The process of claim 20, wherein the at least one butter is between about 70% by weight and about 99% by weight, and the colorant is between about 1% by weight and 30% by weight.

22. The process of claim 21, wherein the at least one butter is between about 80% by weight and about 99% by weight, and the colorant is between about 1% by weight and 20% by weight.

23. The process of claim 18, wherein the colorant comprises a pigment dispersion.

24. The process of claim 22, wherein the pigment dispersion comprises a polysiloxane, wherein the polysiloxane comprises the following chemical structure: 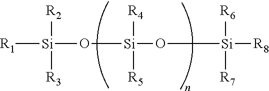
wherein R₁-R₇ are each independently selected from H, —CH₃, —CH₂CH₃, —OH, vinyl; and,
wherein n is an integer from about 3 to about 1,000,000.

25. The process of claim 24, wherein the polysiloxane is dimethylpolysiloxane.

26. The process of claim 25, wherein the pigment comprises an inorganic pigment, an organic pigment, or mixtures thereof.

27. The process of claim 26, wherein the pigment comprises an inorganic pigment selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

28. The process of claim 26, wherein the pigment comprises an organic pigment selected from the group consisting of FD&C Blue #1, FD&C Yellow #5, FD&C Yellow #6, FD&C Yellow #7, FD&C Yellow #8, FD&C Yellow #10, FD&C Red #2, FD&C Red #4, FD&C Red #16, FD&C Red #7, FD&C Red #9, FD&C Red #17, FD&C Red #19, FD&C Red #21, FD&C Red #22, FD&C Red #27, FD&C Red #28, FD&C Red #30, FD&C Red #31, FD&C Red #33, FD&C Red #34, FD&C Red #36, FD&C Red #40, D&C Violet #2, Ext D&C Violet #2, FD&C Green #3, D&C Green #5, D&C Green #6, D&C Green #8, D&C Orange #4, D&C Orange #5, D&C Brown #1, D&C Black #2, carmine, and mixtures thereof.

29. The process of claim 18, wherein the composition further comprises an oil.

30. The process of claim 29, wherein the at least one butter is between about 45% by weight and about 98% by weight, the oil is between about 1% by weight and about 49% by weight, and the colorant is between about 0.05% by weight and 54% by weight.

31. The process of claim 30, wherein the at least one butter is between about 50% by weight and about 80% by weight, the oil is between about 1% by weight and about 35% by weight, and the colorant is between about 1% by weight and 40% by weight.

32. The process of claim 31, wherein the at least one butter is between about 60% by weight and about 78% by weight, the oil is between about 1% by weight and about 25% by weight, and the colorant is between about 1% by weight and 20% by weight.

33. The process of claim 30, wherein the butter is chosen from the group comprising aloe, apricot, avocado, babassu, bacuri, cocoa, coffee, cupuacu, drago, hemp seed, illipe, kokum, macadamia nut, mango, maaracuja, mjochaccino, murumuru, olive, pataua, pistachio nut, shea, shealaoe, sweet almond, ucuuba, wheatgerm, tucuma, lamond, brazil nut, jojoba, pumpkin, soy, chamomile, horestail, lavender, lemon grass, matha grean tea, nanoni, lemon, orange, tangerine, pam, acai, blueberry, cranberry, pomegranate, and mixtures thereof,
wherein the oil is chosen from the group comprising allspice, jamaica, amyris, angelica root, anise seed, armoise, basil, bay, bergamot, black pepper, cajeput, camphor, cananga, cardamom, carrot seed, cassia, cedarleaf, cedarwood, celery seed, cinnamon leaf, cistus, citronella, clary sage, clovebud, coriander, cypress, davana, dill seed, elemi, chamomile, eucalyptus globulus, fennel, sweet, fir, balsam, frankincense, galbanum, garlic, geranium, ginger, grapefruit, white, gurjum balsam, helichrysum italicum, hyssop, juniper berry, lavandin, lavender, lemon, lemongrass, lime, litsea cubeba, lovage, mandarin, marjoram, mullein, myrrh, neroli, niaouli, nutmeg, olibanum, opoponax, orange, palmarosa, patchouli, pennyroyal, peppermint, petitgrain, pine needle, roman chamomile, rose, rosemary, rosewood, sage, sandalwood, spearmint, spikenard, star anise, tangerine, tea tree, thyme, tulsi, valencia, verbena, vetiver, ylang ylang, zdravetz, vanilla, benzoin, tea tree, acai, black raspberry seed, blackberry seed, blackcurrant seed, blueberry seed, cranberry seed, red raspberry seed, seabuckthorn, strawberry seed, algae, almond, aloe vera extract, andiroba, apple seed, apricot kernel, argan, arnica, avocado, baobab, black cumin seed, borage, brazil nut, broccoli seed, buriti fruit, calendula extract, camellia, carrot extract, castor, chardonnay grapeseed, chaulmoogra, cherry kernel, chilean hazelnut, cinnamon leaf, cocoa, coconut, coffee, copaiba balsam, corn, cucumber seed, curry leaf, evening primrose, flax seed, fractionated coconut, grape seed, hazelnut, hemp seed, jojoba, karanja, kiwi seed, kukui nut, macadamia nut, macauba, mango manketti, maracuja, marula, meadowfoam, monoi de tahiti, moringa, neem, olive, palm kernel, palm, papaya, passionfruit oil, pataua, peach kernel, pecan, pequi, perilla seed, pomace olive, poppy seed, pracaxi, pumpkin seed, red palm fruit, rice bran, riesling grapeseed, rosehip, safflower, sangre de drago, saw palmetto, sesame, shea, soybean, st. john's wort, sunflower, tamanu, tomato seed, turkey red, vegetable squalene, vitamin E, walnut, watermelon seed, wheatgerm, yangu, coconut oil, and mixtures thereof.

34. The composition of claim 1, wherein the composition is substantially free from at least one of the group consisting of octyldodecyl neopentanoate, emulsifiers, gums, synthetic thickeners, cyclic methylsiloxanes, gluten, paraben, talc, formaldehyde, mineral oil, petroleum, phthalates, sodium lauryl sulfate, synthetic dye, and synthetic fragrance.

35. The composition of claim 34, wherein the composition is substantially free of octyldodecyl neopentanoate.

36. The composition of claim 1, wherein the composition is substantially free of octyldodecyl neopentanoate, emulsifiers, gums, synthetic thickeners, cyclic methylsiloxanes, gluten, paraben, talc, formaldehyde, mineral oil, petroleum, phthalates, sodium lauryl sulfate, synthetic dye, and synthetic fragrance.

37. A cosmetic composition comprising:
a first component containing palmitic acid, stearic acid, oleic acid, and linoleic acid; and,
a colorant, wherein the composition contains substantially no exogenous water.

38. The composition of claim 37, wherein the composition further comprises:
a second component containing palmitic acid, stearic acid, oleic acid, and linoleic acid, wherein the composition is substantially free from at least one of the group consisting of octyldodecyl neopentanoate, emulsifiers, gums, synthetic thickeners, cyclic methylsiloxanes, gluten, paraben, talc, formaldehyde, mineral oil, petroleum, phthalates, sodium lauryl sulfate, synthetic dye, and synthetic fragrance.

39. The composition of claim 38, wherein the first component is about 40% by weight to about 60% by weight oleic acid, about 20% by weight to about 50% by weight stearic acid, about 3% by weight to about 11% by weight linoleic acid, about 2% by weight to about 9% by weight palmitic acid, and the second component is about 4% by weight to about 9% by weight palmitic acid, about 1% by weight to about 7% by weight stearic acid, about 10% by weight to about 14% by weight oleic acid, and about 48% by weight to about 74% by weight linoleic acid.

40. The composition of claim 39, wherein the composition further comprises:
a third component containing about 7% by weight palmitic acid, about 4% by weight stearic acid, about 16% by weight oleic acid, less than about 1% by weight alpha-linoleic acid, less than about 1% by weight palmitoleic acid, about 0.8% by weight to about 1.5% by weight tocopherols and steroids, and about 72% by weight linoleic acid; and,
a fourth component containing about 66% by weight to about 71% by weight eicosenoic acid, about 14% by weight to about 20% by weight docosenoic, and about 10% by weight to about 13% by weight oleic acid.

41. The composition of claim 40, wherein the composition is substantially free of octyldodecyl neopentanoate, emulsifiers, gums, synthetic thickeners, cyclic methylsiloxanes, gluten, paraben, talc, formaldehyde, mineral oil, petroleum, phthalates, sodium lauryl sulfate, synthetic dye, and synthetic fragrance.

42. The composition of 13, wherein the composition further comprises a wax.

43. The composition of 29, wherein the composition further comprises a wax.

44. The composition of 38, wherein the composition further comprises a wax.
